(19) United States
(12) Patent Application Publication
(10) Pub. No.: US 2025/0134078 A1
(43) Pub. Date: May 1, 2025

(54) TRANSGENIC MOUSE

(71) Applicant: Seiho NAGAFUCHI, Fukuoka-shi, Fukuoka (JP)

(72) Inventors: Seiho NAGAFUCHI, Fukuoka-shi, Fukuoka (JP); Keiichiro MINE, Saga-shi, Saga (JP)

(21) Appl. No.: 18/262,566

(22) PCT Filed: Feb. 17, 2023
(86) PCT No.: PCT/JP2023/005817

§ 371 Date: Dec. 11, 2024

(30) Foreign Application Priority Data
Mar. 4, 2022 (JP) 2022-033681

(51) Int. Cl. A01K 67/0278; C07K 14/47

(52) U.S. Cl. CPC A01K 67/0278; C07K 14/47; A01K 2217/072; A01K 2227/105; A01K 2267/0362

(57) ABSTRACT

Provided is a transgenic mouse that expresses a human coxsackie group B virus receptor gene specifically in pancreatic beta cells. The transgenic mouse has DNA encoding a human coxsackie group B virus receptor in pancreatic beta cells and expresses the human coxsackie group B virus receptor specifically in the pancreatic beta cells.

### TECHNICAL FIELD

The present invention relates to a transgenic mouse, and more particularly, to a transgenic mouse with a virus receptor encoded.

### BACKGROUND ART

Regarding diabetes, the Ministry of Health, Labor and Welfare's estimate in 2016 is publicly available, reporting that the number of Japanese diabetes patients is 10 million, and if the number of potential diabetes patients is included, it reaches as many as 20 million. Further, the International Diabetes Federation (IDF) has warned that the number of world diabetes patients is 463 million in 2019 and will reach 700 million in 2045.

Such prevalence and increase in number of diabetes patients both in Japan and overseas are not only a social problem but also an important issue affecting individual health, economy, and quality of life. Diabetes is understood to be greatly influenced by overeating, obesity, lack of exercise, and aging as one of lifestyle-related diseases accompanying economic progress in society, while the importance of viral infection is also being recognized.

Many basic and clinical studies have been accumulated so far on virus-induced diabetes, and it is now known that various viruses are involved in the onset of diabetes. Candidate viruses include, for example, coxsackie group B (CoxB) viruses, hepatitis A virus, rubella virus, mumps virus, and rotavirus. Further, there have been reports one after another that the novel coronavirus is also involved in the onset of diabetes.

There are currently accumulated a number of reports that coxsackie group B (CoxB) viruses, which belong to enteroviruses, among diabetes-causing candidate viruses, are found in pancreatic islets of patients with acute onset diabetes (see, for example, Non-Patent Literatures 1 and 2).

Further, there are reports that isolated viruses cause diabetes in experimentally immunity-weakened mice (see, for example, Non-Patent Literatures 3, 4, and 5).

Under these circumstances, studies on transgenic animal models intended to develop diabetes have been actively pursued. For example, such a conventional transgenic animal model is known that expresses a herpesvirus receptor CD46 in monkey central nervous system cells and develops diabetes by herpesvirus infection (see, for example, Patent literature 1). However, this is not the case that the expression occurs in pancreatic beta cells. Furthermore, this does not target mice, which are the most widely used animal models.

In this regard, a diabetes onset transgenic mouse in which human HB-EGF cDNA is placed downstream of a human insulin promoter is known as a conventional transgenic animal model using a receptor to be expressed in pancreatic beta cells (see, for example, Patent Literature 2). However, this is not the case that the above-mentioned coxsackie group B (CoxB) virus receptor is expressed in mouse pancreatic islet cells.

Here, the inventors have reported on their preliminary research to the effect that they challenged production of a transgenic mouse in which a human coxsackie and adenovirus receptor (hCXADR), which is a receptor for a coxsackie group B (CoxB) virus, is expressed in mouse pancreatic islet cells, confirmed gene transfer, and calculated transgene copy number (see Non-Patent Literature 6). However, this is only a preliminary research content, and in fact, a desired transgenic mouse was not completed at that time.

The inventors have also tried to produce a model mouse capable of detecting viral diabetes inducibility by: creating a mouse that produces a substance that facilitates attachment of a candidate diabetes-inducing virus to the surface of pancreatic islet cells; confirming establishment of infection with human coxsackie group B virus in the mouse; and crossing it with a mouse having multiple viral diabetes susceptible genes that could be obtained so far. However, the produced model mouse did not develop diabetes when attacked with the coxsackie group B virus (see Non-Patent Literature 7).

### CITATION LIST

### Patent Literature

Patent Literature 1: Japanese Translation of PCT International Application Publication No. JP-T-2008-523785
Patent Literature 2: Japanese Translation of PCT International Application Publication No. JP-T-2009-527217

### Non-Patent Literature

Non-Patent Literature 1: Jimbo E, Kobayashi T, Takeshita A, Mine K, Nagafuchi S, Fukui T, Yagihashi S. Immunohistochemical detection of enteroviruses in pancreatic tissues of patients with type 1 diabetes using a polyclonal antibody against 2A protease of Coxsackievirus. J Diabetes Investigation published online.
Non-Patent Literature 2: Nagafuchi S (Guest Editor). Editorial: Regulation of viral infection in diabetes. Biology 10:529, 2021.
Non-Patent Literature 3: Nagafuchi S, Mine K, Takahashi H, Anzai K, Yoshikai Y. Viruses with masked pathogenicity and genetically susceptible hosts. J Med Virol 91:1365-1367, 2019 with August Issue Front Cover.
Non-Patent Literature 4: Flodström M, Maday A, Balakrishna D, Cleary M M, Yoshimura A, Sarvetnick N. Target cell defense prevents the development of diabetes after viral infection. Nat Immunol. 3:373-382, 2002.
Non-Patent Literature 5: McCartney S A, Vermi W, Lonardi S, Rossini C, Otero K, Calderon B, Gilfillan S, Diamond M S, Unanue E R, Colonna M. RNA sensor-induced type I IFN prevents diabetes caused by a β cell tropic virus in mice. J Clin Invest. 121:1497-1507, 2011.
Non-Patent Literature 6: https://kaken.nii.ac.jp/ja/grant/KAKENHI-PROJECT-18H02853/
Non-Patent Literature 7: https://japan-iddm.net/wpcontent/uploads/grant/report/013_SagaUniv_Nagafuchi.pdf

### SUMMARY OF INVENTION

### Technical Problem

Thus, such a transgenic mouse that can be used as the model mouse capable of detecting viral diabetes inducibility has not been obtained yet.

As shown in Non-Patent Literatures 4 and 5, there are reports that isolated viruses cause diabetes in mice having experimentally weakened immunity, but in fact there is little conclusive evidence.

The reason for this is that there is still no suitable test system for viral diabetes inducibility (an animal model that accurately mimics the pathogenic mechanism of human viral diabetes) that satisfies Koch's three postulates (1. A certain microorganism must be found in a certain disease. 2. Such a microorganism must be isolated. 3. The isolated microorganism should cause the same disease when introduced into a susceptible animal.), which is a method for proving pathogens.

That is, in Koch's postulates, a proof that a certain microorganism is a cause of a certain disease is on the basis that an animal model (e.g., mouse) that mimics human susceptibility is used to develop such a disease. However, there is no such animal model for diabetes, so that Koch's postulates cannot be in fact confirmed.

Mouse pancreatic islet cells, which are most commonly used as animal models, lack the expression of a receptor (hCXADR) for a human coxsackie group B virus (human CoxB virus), and thus have poor susceptibility to infection with the human coxsackie group B virus and cannot be used as a validation model for human viral diabetes as it is.

As described in Non-Patent Literature 6 above, the inventors have reported that they challenged production of the transgenic mouse that expresses the human coxsackie and adenovirus receptor (hCXADR) in mouse pancreatic islet cells, and calculated a transgene copy number at a certain expression level.

However, the expression level of gene transfer (transgene) does not in general correlate with the copy number of the introduced genes (see Examples below). According to the common general knowledge in the art, in general, even if gene transfer results in some expression level, this does not necessarily lead to the completion of a transgenic mouse.

In fact, the inventors have not obtained evidence for the completion of the transgenic mouse that expresses the human coxsackie and adenovirus receptor (hCXADR) in mouse pancreatic islet cells, as shown in Non-Patent Literature 6. In other words, also in Non-Patent Literature 6, such an excellent transgenic mouse has not been completed yet, and thus a transgenic mouse that develops specific pathologies such as diabetes has not been confirmed yet.

In this way, there is a strong need for a model mouse to be used for identifying human coxsackie group B virus (human CoxB virus), which is a promising virus involved in the onset of diabetes, based on scientific grounds. At present, however, there is a problem that no model mouse with such a strong need exists.

The present invention has been made to solve the above problem. It is an object of the present invention to provide a transgenic mouse that expresses a human coxsackie group B virus receptor (hCXADR) gene specifically in pancreatic beta cells.

### Solution to Problem

To achieve the above object, the inventors have succeeded in creating a transgenic mouse designed to make mouse pancreatic beta cells susceptible to infection with human coxsackie group B viruses and derived the present invention.

Thus, the present invention provides a transgenic mouse having DNA that encodes a human coxsackie group B virus receptor in pancreatic beta cells and expressing the human coxsackie group B virus receptor (hCXADR) specifically in the pancreatic beta cells.

The transgenic mouse according to the present invention highly expresses the human coxsackie group B virus receptor (hCXADR) specifically in the pancreatic beta cells and develops diabetes (see Examples below), thus making it possible to efficiently promote research on the relationship between the coxsackie group B virus and diabetes. This transgenic mouse can be used as a model animal for performing, for example, validation of new vaccines on diabetes and medicines effective for humans, thereby obtaining excellent effects such as being available for the development of new therapeutic drugs. The present invention also provides a method for producing a preventive vaccine against diabetes using this transgenic mouse and a method for screening a therapeutic drug for diabetes using this transgenic mouse.

### BRIEF DESCRIPTION OF DRAWINGS

FIG. 1 shows a base sequence of DNA that encodes a receptor for a coxsackie group B virus (human coxsackie and adenovirus receptor), hCXADR, with respect to a transgenic mouse according to a first embodiment of the present invention;

FIG. 2 is a flowchart of a method for producing a diabetes preventive vaccine using the transgenic mouse according to the first embodiment of the present invention;

FIGS. 3A and 3B describe a mouse insulin promoter (MIP) used for creating a transgenic mouse according to Example 1 of the present invention;

FIG. 4 shows an example of a pUC-based plasmid vector map including the MIP used for creating the transgenic mouse according to Example 1 of the present invention;

FIG. 5 shows an example of a plasmid vector map of MIP-hCXADR used for creating the transgenic mouse according to Example 1 of the present invention;

FIG. 6 describes a virus receptor (hCXADR) gene to be injected into a mouse fertilized egg used for creating the transgenic mouse according to Example 1 of the present invention;

FIG. 7 is a schematic diagram of a MIP-hCXADR plasmid vector used for creating the transgenic mouse according to Example 1 of the present invention;

FIG. 8 shows a check (PCR method) result of successful gene transfer for mice born from transgenic fertilized eggs in the transgenic mouse according to Example 1 of the present invention;

FIG. 9 shows a calculation result of a transgene copy number of a coxsackie and adenovirus receptor (hCXADR) gene using Southern blotting (DNA/DNA hybrid method) in the transgenic mouse according to Example 1 of the present invention;

FIG. 10 shows a relative expression level and a transgene copy number of a human coxsackie and adenovirus receptor (hCXADR) gene for each individual in the transgenic mouse according to Example 1 of the present invention;

FIGS. 11A to 11C show results of immunostaining of hCXADR-positive mouse pancreatic islets infected with a coxsackie B4 virus in the transgenic mouse according to Example 1 of the present invention;

FIG. 12 shows a relative expression level of a human coxsackie and adenovirus receptor (hCXADR) gene for each organ in a transgenic mouse according to Example 2 of the present invention;

FIGS. 13A and 13B show resultant images of pancreatic islet tissue seven days after intraperitoneal inoculation of a coxsackie B4 virus for the transgenic mouse (line 5) according to Example 2 of the present invention;

FIG. 14 shows a change in blood glucose level (mg/dl) after attack (infection) with the coxsackie B4 virus for a transgenic mouse according to Example 3 of the present invention; and

FIGS. 15A and 15B shows confirmation results of protection against infected pancreatic islet damage after attack (infection) with the coxsackie B virus depending on the presence or absence of a coxsackie B virus vaccine according to Example 4 of the present invention.

### DESCRIPTION OF EMBODIMENTS

### First Embodiment

A transgenic mouse according to this embodiment has DNA encoding a human coxsackie group B virus receptor (hCXADR) in pancreatic islet cells and expresses the human coxsackie group B virus receptor specifically in pancreatic beta cells.

This human coxsackie virus group B virus receptor is applicable to any of types 1, 2, 3, 4, 5, and 6 of the human coxsackievirus group B.

This human coxsackie group B virus receptor is not limited and may be any receptors that act as a receptor for a human coxsackie group B virus. For example, a human Coxsackie and Adenovirus receptor (hCXADR(hCAR)) or a human Decay Accelerating Factor (DAF) can be used. In addition, any other receptors that act as the receptor for the human coxsackie group B virus may be used.

Abbreviations hCXADR and hCAR are known as an abbreviation for this human coxsackie and adenovirus receptor, and both of them can be used. The abbreviation hCXADR, which has been internationally recognized and widely accepted, will be used herein for convenience to avoid confusion with CAR (Chimeric Antigen Receptor), which is also used in another medical field. That is, the human coxsackie adenovirus receptor referred to herein as hCXADR is synonymous with the human coxsackie adenovirus receptor referred to as hCAR.

The transfer site of DNA encoding this human coxsackie group B virus receptor (hCXADR) can be placed under control of a mouse insulin promoter (MIP).

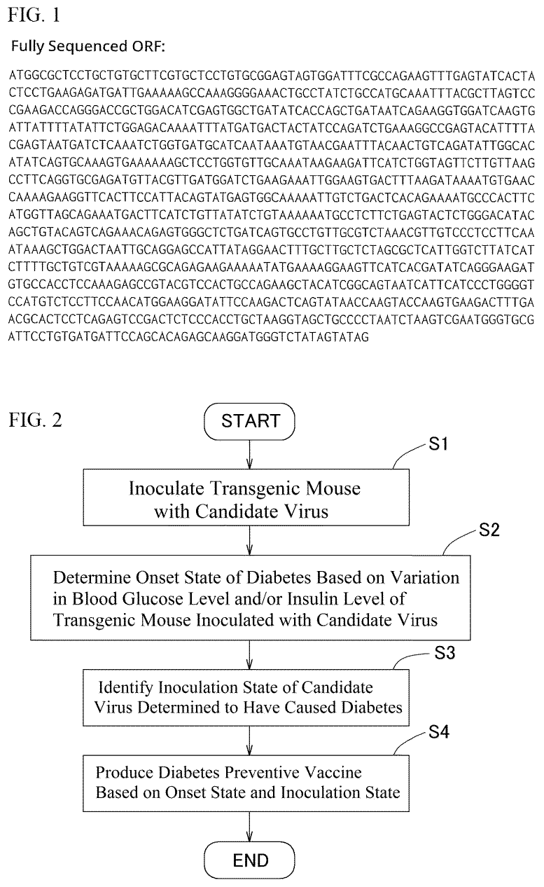

The DNA encoding this human coxsackie and adenovirus receptor (hCXADR) may be a polynucleotide comprising a nucleotide sequence as shown in FIG. 1, or a polynucleotide having a nucleotide sequence identity of 90% or more to the polynucleotide comprising the nucleotide sequence as shown in FIG. 1.

The DNA encoding this human coxsackie and adenovirus receptor (hCXADR) can be used by purchasing a commercial product (for example, manufactured by OriGene).

A gene for expressing this human coxsackie and adenovirus receptor (hCXADR) specifically in mouse pancreatic beta cells can be, but not particularly limited to, a vector for gene transfer (gene carrier) in which a human coxsackie and adenovirus receptor (hCXADR) gene is bound to the mouse insulin promoter (MIP). Such a vector may include a plasmid vector that propagates in bacteria. In one example, a pUC-based plasmid vector (for example, pTimer-1 Vector: Clontech) can be used, although other various plasmid vectors can be used.

For example, the plasmid vector may be a plasmid vector composed of the mouse insulin promoter comprising a linear DNA fragment containing an SV40 polyA signal and the human coxsackie and adenovirus receptor (hCXADR).

A rat insulin promoter (RIP) can be used as an alternative to the mouse insulin promoter.

It has been confirmed that the transgenic mouse of the present embodiment thus obtained causes the human coxsackie and adenovirus receptor (hCXADR) gene to be expressed specifically in pancreatic beta cells of mouse pancreatic islets, with a high degree of its expression (see Examples below).

In addition, the transgenic mouse of the present embodiment may be produced by other methods than the above. For example, it can be produced in wild-type mouse or genetically-modified mouse backgrounds.

Such a transgenic mouse that expresses the human coxsackie group B virus receptor (hCXADR) gene in the insulin-producing pancreatic beta cells is previously unknown, and thus can be regarded as a novel diabetes model animal.

A method for evaluating the transgenic mouse of the present embodiment is not limited and may include, for example, inoculating the transgenic mouse of the present embodiment with formalin-inactivated coxsackie B virus and ten days later with coxsackie B virus to evaluate damage of pancreatic beta cells and an increase in blood glucose. It has been in fact confirmed that the transgenic mouse of the present embodiment shows the damage of pancreatic islet cells and the increase in blood glucose when attacked by the coxsackie B virus (see Examples below).

Diabetes for which the transgenic mouse of the present embodiment is to be used can be either type 1 or type 2. That is, it has been pointed out that viruses are involved in the onset of both types of diabetes, and thus both type 1 diabetes and type 2 diabetes can be used.

Since the transgenic mouse of the present embodiment exhibits pathology of diabetes due to viral infection as described above, using the transgenic mouse of the present embodiment allows production of diabetes preventive vaccines and screening of diabetes therapeutic drugs.

An example of a method for producing a diabetes preventive vaccine is shown in FIG. 2. First, the transgenic mouse is inoculated with a candidate virus (S1: inoculation step). Such a candidate virus includes, for example, human coxsackie group B (CoxB) virus that is suggested to potentially cause diabetes. The human coxsackie group B (CoxB) virus belongs to family Picornaviridae, like a poliovirus, and is broadly included in genus Enterovirus. Regarding this genus Enterovirus, vaccines against enteroviruses A6, A9, A10, A16, and A71, which are major causes of hand-foot-and-mouth disease, and enteroviruses D68 and D71, which cause nerve paralysis, are currently under development both in Japan and overseas.

Next, an onset state of diabetes is determined based on variation in blood glucose level and/or insulin level of the transgenic mouse inoculated with the candidate virus (S2: determination step). Such an onset situation can be determined on the basis of, for example, whether a blood glucose level and/or an insulin level of the transgenic mouse exceeds a threshold, and/or whether a fluctuation range(s) thereof exceeds a threshold.

Next, an inoculation state of the candidate virus determined to have caused diabetes is identified (S3: identification step). The inoculation state is determined by comprehensively reflecting, for example, the inoculation amount, inoculation concentration, and inoculation time interval of the candidate virus inoculated into the transgenic mouse.

A diabetes preventive vaccine is produced based on the onset state and the inoculation state (S4: production process). These obtained onset state and inoculation state clarify various conditions of the candidate virus required for use in the diabetes preventive vaccine, thus making it possible to produce an optimal diabetes preventive vaccine.

The type of vaccine that can be produced is not limited, and known techniques can be used. Such techniques include classical formalin-inactivated vaccines to viral protein vaccines, and recently developed mRNA vaccines, which are currently in practical use. These can be selected appropriately depending on their intended use.

A vaccination program for the obtained diabetes preventive vaccine according to the present embodiment is not limited. However, since the human coxsackie group B (CoxB) virus is included in the genus Enterovirus like a poliovirus as described above, it is preferable to carry out the vaccination program according to a poliovirus vaccine.

A preferable example of the vaccination program for the diabetes preventive vaccine according to the present embodiment is to carry out vaccinations at the same timing as that of three vaccinations at three-to-eight-week intervals from three months after birth, and the fourth vaccination at six months to one year after the third vaccination, or the timing equivalent thereto, as with the poliovirus vaccine.

In addition, such a vaccination program is also possible that carries out three vaccinations at three-to-eight-week intervals from three months after birth, and the fourth vaccination at six months to one year after the third vaccination, according to the poliovirus vaccination schedule.

Furthermore, the diabetes preventive vaccine according to the present embodiment can also be formed as a multivalent vaccine composed of a plurality of vaccines together with poliovirus. For example, the diabetes preventive vaccine according to the present embodiment can comprehensively include one or more enteroviruses such as A6, A9, A10, A16, A71, and D68 to form a “Comprehensive Polyvalent Polio-Enterovirus Vaccine.”

The vaccination program for this multivalent vaccine can also be such a vaccination program that carries out three vaccinations at three-to-eight-week intervals from three months after birth, and the fourth vaccination at six months to one year after the third vaccination, according to the case of poliovirus vaccine mentioned above.

Subjects to be vaccinated with the diabetes preventive vaccine according to the present embodiment are not limited. However, it is preferable to target all young people for vaccination, since each of the above-mentioned enteroviruses, including the human coxsackie group B (CoxB) virus, tends to infect humans widely during childhood.

The effect of the diabetes preventive vaccine according to the present embodiment is estimated as follows in Japan. Based on the fact that pneumococci are about 20% effective against pneumonia in elderly people who are sick with bacteria or viruses, one can think that it is effective in one fifth (⅕) of the estimated number of people affected with diabetes due to viruses.

Based on the above, a preventive effect on type 1 diabetes (total damage to insulin-producing pancreatic beta cells) patients is estimated to be found in about 100 patients per year for the following reasons. The incidence of type 1 diabetes for young people is considered to be more than 2 per 100,000 people. Since type 1 diabetes occurs at all ages, the number of affected people is estimated to be between 2,000 and 3,000 (about 2,500) people per year. According to the inventors' research report (see EBioMedicine, 2015), about 20% of affected people have, in general, symptoms of infection at the time of onset. This calculates that the number of affected people due to infection is about 500 people, and that one fifth (⅕) of them are protected by the vaccine.

Further, a preventive effect on type 2 diabetes (partially damage to pancreatic beta cells) patients is estimated to be found in about 1,000 patients per year for the following reasons. About 10% of patients with type 2 diabetes have a virus susceptibility gene (TYK2 promoter variant) (see EBioMedicine, 2015 mentioned above). About 100,000 Japanese people develop type 2 diabetes each year, and about 10,000 of them are estimated to have the susceptibility gene of TYK2 promoter variant. These carriers are twice as likely to develop type 2 diabetes. Thus, it is calculated that about 5,000 people are at risk of developing this disease due to viral infection, and the preventive effect of the vaccine is one fifth (⅕).

For international vaccine efficacy estimates, it is understood that viral diabetes is typically involved in the development of type 1 diabetes. The annual incidence of type 1 diabetes in Japan is estimated to be about 2.5 per 100,000 people, which is the lowest level in the world. On the other hand, the annual incidence of type 1 diabetes in other countries is reported to be 52.2 per 100,000 people in Finland and 28.1 per 100,000 people in the UK (see Diabetes Atlas, International Diabetes Federation, 2021).

Although a percentage of people affected with type 1 diabetes caused by viruses is unknown, it is reasonable to estimate that viral infection is a trigger for the onset of about 20% of type 1 diabetes patients (the percentage of people with symptoms of infection at the time of onset) in Japan. Thus, the preventive effect of the diabetes preventive vaccine according to the present embodiment is valid.

According to a recent report from the International Diabetes Federation, the number of diabetes patients in the world is 790 million, most of whom suffer from type 2 diabetes, and an increase of 8 million is expected each year (see International Diabetes Federation, Diabetes Atlas, pp. 1-142, 2021). The involvement of viral infection in type 2 diabetes has not been internationally reported so far due to the difficulty of its evidence. However, the inventors could identify the viral diabetes susceptible gene and find out that its percentage for type 2 diabetes patients (prevalence rate of about 10%) is twice that for healthy controls (prevalence rate of about 5%), thereby reporting that its risk is also approximately doubled (see EBioMedicine, 2015). Furthermore, insulin secretion was found to be decreased in type 2 diabetes patients having such susceptible gene, suggesting that viral infection causes pancreatic islet cell damage (see Genes, 2021).

The number of world diabetic patients is increasing by about 8 million each year, most of which are considered to suffer from type 2 diabetes. Further, it has been experimentally confirmed that the diabetes preventive vaccine according to the present embodiment can significantly protect pancreatic islets from infection damage caused by the coxsackie B virus after its vaccination, that is, it has the effect of suppressing the onset of diabetes (see Examples below). Also based on the above, the implementation of the diabetes preventive vaccine according to the present embodiment can greatly reduce the incidence of diabetes, which is one of the major health problems in the world.

In addition, a method for screening a therapeutic drug for diabetes is not limited, and known methods can be used. Such methods include, for example, a relatively simple and convenient method of administering an infected candidate antiviral agent and validating its effect.

The present invention will be described in more detail below with reference to Examples. However, the present invention is not limited to the following Examples.

### Example 1

### (Production of Transgenic Mouse)

### (1) Sequence

### (1-1) Mouse Insulin Promoter (MIP)

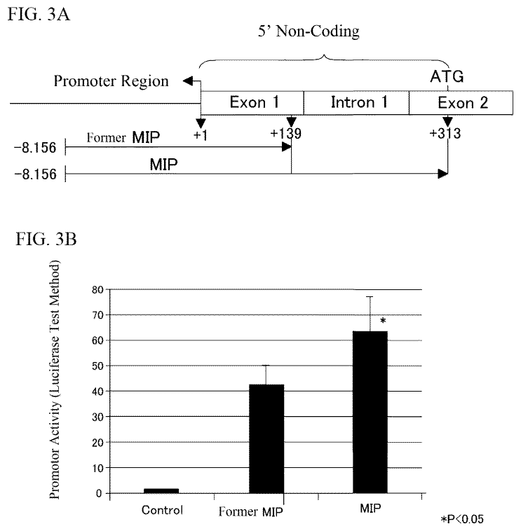

As shown in FIG. 3A, this is genomic DNA, 8,469 bp, identified, and a sequence of an upstream region encoding a mouse insulin. This is a sequence ranging between −8,156 bp upstream and 313 bp downstream from a transcription start point, totaling 8,469 bp, and includes a 5′ non-coding region (Exon 1, Intron 1, and Exon 2 up to just before ATG) up to a translation start point. This is not related to pathogenicity or transmissibility to animals such as mammals.

The mouse insulin promoter (MIP) is known to activate its subsequent genes (see Hara M, et al. Am J Physiol Endocrinol Metab, 2003). The inventors have confirmed, using this MIP gene (former MIP), that its function works (see Izumi K, Mine K, Nagafuchi S, et al. Nat Commun, 2015).

The MIP shown in FIG. 3A is configured to have +139 to +313 added compared to the former MIP previously used, and include a promoter region (−8.156) as well as Exon 1, Intron 1, and Exon 2 up to +313 (just before ATG). Thus, such a new and more active mouse insulin promoter is herein referred to as MIP to clearly distinguish it from the former MIP previously used.

Extending the scope of genes in this way increased the activity of the new and highly active mouse insulin promoter MIP (P<0.05) more than that of the former MIP as shown in FIG. 3B, which was confirmed from a more detailed study of the function of such MIP.

(1-2) Human Coxsackie Virus and Adenovirus Receptor (hCXADR)

This is cDNA, 1.1 kb, identified (NM_001338.3, manufactured by OriGene), and a sequence encoding a coxsackie virus receptor and an adenovirus receptor (see FIG. 1).

(2) Cloning of Human Coxsackie and Adenovirus Receptor (hCXADR) Gene

A purchased commercial product (manufactured by OriGene) of human coxsackie virus and adenovirus receptor (hCXADR), cDNA [SC108081: Human coxsackie virus and adenovirus receptor (hCXADR), transcript variant 1, NM_001338.3], was used to amplify with the PCR method a region encoding hCXADR gene proteins and replace it with the DsRed1-E5 sequence of the pUC-based plasmid vector containing MIP (a plasmid vector in which only MIP is inserted) (pTimer-1 Vector: Clontech) as shown in FIG. 4 for cloning. The plasmid DNA (pUC-based) used for the cloning of MIP-hCXADR is derived from Escherichia coli (Enterobacteriaceae Escherichia coli, class 1), and there is no possibility of exchanging or carrying out donor nucleic acids in transmissibility. Plasmids are genes that self-replicate in bacteria and that are used to propagate various genes.

### Description of Words and Phrases in FIG. 4

MIP: mouse insulin promoter
DsRed1-E5
MCS: multiple cloning site
SV40 poly A⁺: SV40 early poly A signals
f1 ori P: f1 origin of replication
P: Promoter for Kan^{r}
SV40 ori: SV40 origin of replication
P_{SV40}: SV40 early promoter and enhancer
Kan^{r}/Neo^{r}: kanamycin/neomycin resistance gene
HSV TK poly A⁺: Herpes simplex virus thymidine kinase polyadenylation signals
pUC ori: origin of replication

As shown in the map of the pUC-based plasmid vector containing MIP shown in FIG. 4 above, a promoter or promoter/enhancer sequence was inserted into the multiple cloning site upstream of the sequence encoding the fluorescent timer protein DsRed1-E5, and transcription from the promoter was monitored. The pUC-based plasmid vector containing this MIP has a kanamycin/neomycin resistance gene, HSV TKpolyA, DsRed1-E5, SV40 polyA signal, f1 origin, SV40 promoter, and SV40 origin, and has the MIP shown in FIG. 3A inserted into the multiple cloning site.

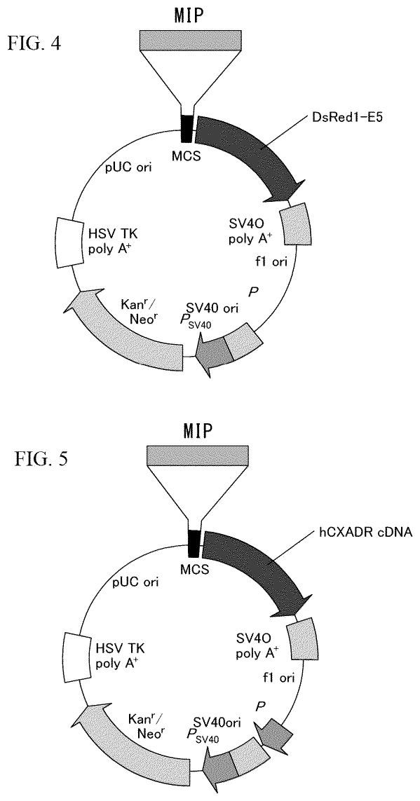

It is possible to increase a target gene by introducing a MIP-hCXADR gene into a plasmid and propagating the plasmid. The increased MIP-hCXADR gene can be taken out to be available for the next step. As shown in the plasmid vector map of MIP-hCXADR in FIG. 5, the DsRed1-E5 portion shown in FIG. 4 above was cleaved with restriction enzyme AgeINotI, and hCXADR cDNA was inserted. As shown in this FIG. 5, a plasmid vector having a MIP-hCXADR-SV40 polyA signal sequence (MIP-hCXADR plasmid vector) capable of expressing the human coxsackie and adenovirus receptor (hCXADR) gene under control of the mouse insulin promoter (MIP) was produced (P1 level).

### Description of Words and Phrases in FIG. 5

MIP: mouse insulin promoter
hCXADR: human coxsackie and adenovirus receptor
MCS: multiple cloning site
SV40 poly A⁺: SV40 early poly A signals
f1 ori P: f1 origin of replication
P: Promoter for Kan^{r}
SV40 ori: SV40 origin of replication
P_{SV40}: SV40 early promoter and enhancer
Kan^{r}/Neo^{r}: kanamycin/neomycin resistance gene
HSV TK poly A⁺: Herpes simplex virus thymidine kinase polyadenylation signals
pUC ori: origin of replication

### (3) Production of Transgenic Mouse

(3-1) Injection of Virus Receptor (hCXADR) Gene into Mouse Fertilized Egg

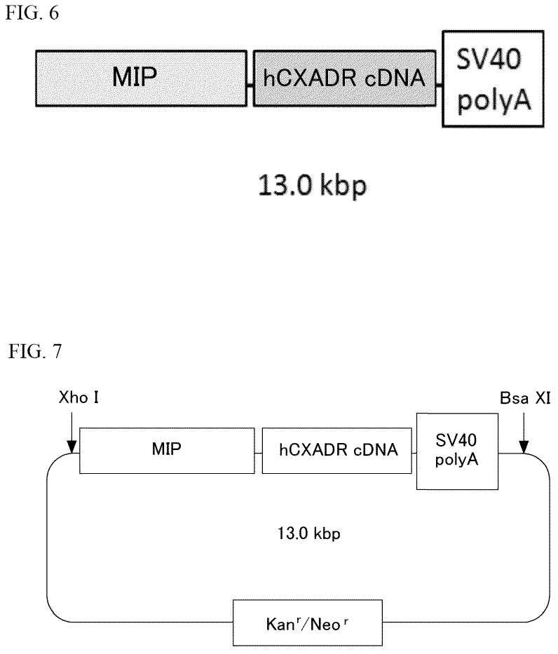

FIG. 6 shows the structure of the virus receptor (hCXADR) gene introduced into the mouse in the plasmid. FIG. 7 is a schematic diagram of the MIP-hCXADR plasmid vector, and indicates by arrows sites where restriction enzyme cleaves the MIP-hCXADR gene introduced into the plasmid (vector: pTimer-1 Vector: Clontech: already mentioned). As this restriction enzyme, XhoI and BsaXI was used to obtain a linear DNA fragment containing the MIP-hCXADR SV40 polyA signal for injection into fertilized eggs.

As shown in FIG. 6, a transgene (see FIG. 6) in which the human coxsackie and adenovirus receptor (hCXADR) is bound to the mouse insulin promoter (MIP) cleaved off from the MIP-hCXADR plasmid vector shown in FIG. 7 above by the restriction enzymes was used as the gene for expressing the human coxsackie and adenovirus receptor specifically in the mouse pancreatic beta cells.

As described above, a transgenic mouse was produced by cleaving the sites of specific gene sequences recognized by the respective restriction enzymes Xhl I and Bsa XI to obtain the MIP-hCXADR gene for the purpose of instruction into mice, and producing a vector having the hCXADR cDNA inserted between the MIP and SV40 polyA signal on the plasmid DNA, and cleaving off, from this produced MIP-hCXADR plasmid, a linear DNA fragment (transgene) containing the MIP-hCXADR-SV40 polyA signal to be injected into fertilized eggs.

### (3-2) Progress of Transgenic Mouse Production

The transgene was injected into a total of 690 fertilized eggs and 580 fertilized eggs started division and growth. These 580 fertilized eggs were transplanted into 24 foster mother female mice and 14 mice of them became pregnant. Subsequently, 25 newborn mice were born and 14 mice of them were living. Of these 14 living mice, 4 mice succeeded in gene transfer. Further, mating was done four times in total, since if genes are integrated into multiple chromosomes in one mouse, subsequent analysis will be complicated. Finally, 5 lines of gene integration positive mice were obtained.

### (3-3) Screening by Gene Amplification (PCR) Test

Next, screening was done with gene amplification (PCR) test to determine whether the gene had been introduced into the born mice and confirm the mice with successful gene transfer. FIG. 8 shows a check (PCR method) result of successful gene transfer for the mice born from the transgenic fertilized eggs. The data shown in FIG. 8 is a result obtained by a specific gene amplification method (PCR) (MIP-hCXADR PCR product, size: 2.5 kb). It was confirmed that sample numbers 2, 3, 7, 8, and 14 were positive.

### (3-4) Southern Blotting Test

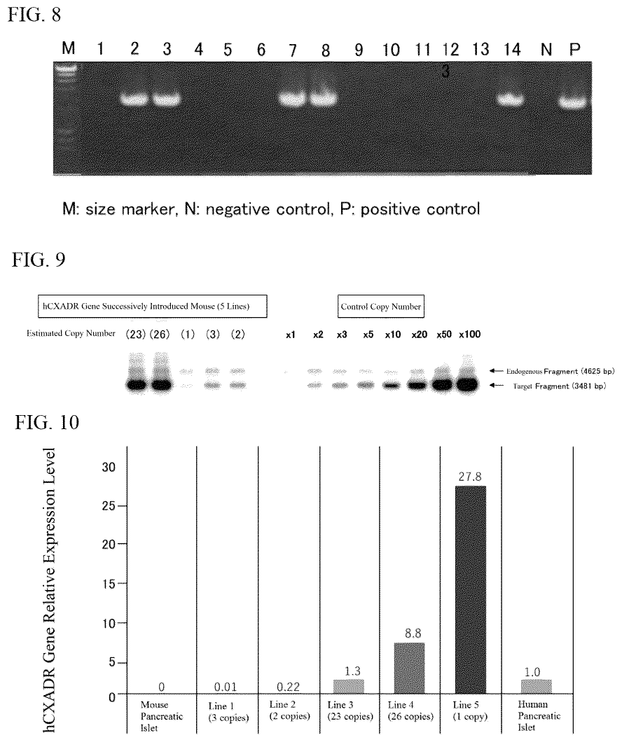

The copy number of the successfully introduced genes was tested by Southern blotting. Using Southern blotting (DNA/DNA hybrid method) in this created transgenic (Mouse Insulin Promoter: MIP-hCXADR Tg) mouse, the transgene copy number of the coxsackie and adenovirus receptor (hCXADR) gene was calculated, whose result is shown in FIG. 9 together with control copy number. Southern blotting is a method that uses a labeled marker gene specifically binding to the introduced gene to detect the number (copy number) of the introduced genes in accordance with the degree of that binding. From the result shown in FIG. 9, it was confirmed that the coxsackie and adenovirus receptor (hCXADR) gene was introduced into the obtained transgenic mice.

Furthermore, FIG. 10 shows a result of comparing a hCXADR gene expression level and a transgene copy number in each pancreatic islet for five mouse lines obtained by hCXADR gene transfer, with those in human pancreatic islet. In particular, FIG. 10 shows a result of detecting the hCXADR gene expression level in each islet cell by quantitative gene amplification (quantitative PCR), assuming that the expression level for the human islet is 1.0. FIG. 10 shows that in the multiple lines 1-5, which can be divided into three categories of low, moderate, and high expression levels, the human coxsackie and adenovirus receptor (hCXADR) gene is expressed specifically in pancreatic beta cells of mouse pancreatic islets. Accordingly, it could be also confirmed that it is expressed specifically in pancreatic islets (organ).

In this way, since the receptor expression level in pancreatic islet cells for humans is highly likely to fluctuate due to, for example, individual differences and some kind of stimuli, 5 lines of transgenic mice (hCXADR Tg mice) could be obtained for the purpose of establishing multiple lines of three categories of low, moderate, and high expression levels.

Furthermore, the inventors studied in detail association between the hCAR gene expression level and the transgene copy number in each pancreatic islet cell for the 5 mouse lines with successful gene transfer.

As shown in FIG. 10, the expression level and the copy number of the introduced hCXADR gene in each line were as follows: expression level 0.01 (copy number 3) for line 1; expression level 0.22 (copy number 2) for line 2; expression level 1.3 (copy number 23) for line 3; expression level 8.8 (copy number 26) for line 4; and expression level 27.8 (copy number 1).

When this result is arranged on the basis of the hCXADR gene expression level for human pancreatic islets, lines 5 and 4, which have a higher expression level than the human pancreatic islets, have copy numbers of 1 and 26, respectively. Line 3, which has almost the same expression level as the human pancreatic islets, has a copy number of 23. Lines 1 and 2, which have a lower expression level than the human pancreatic islets, have copy numbers of 3 and 2, respectively.

That is, it was certainly confirmed that a high and low of the gene transfer (transgene) expression level does not correlate with the copy number of the introduced hCXADR gene. In other words, the steps until the conformation of the expression of gene transfer (transgene) are not determined to be a stage at which the transgenic mouse with the hCXADR gene introduced thereinto has been completed, just as common technical knowledge.

According to such common technical knowledge, the stage of simply introducing human coxsackie and adenovirus receptor (hCXADR) into mouse pancreatic islet cells does not indicate the completion of the transgenic mouse, as shown in Non-Patent Literature 6 mentioned above. This is consistent with the fact that the inventors had not actually completed the transgenic mouse, which is described in Non-Patent Document 6 mentioned above.

As described above, the present embodiment has confirmed the creation of transgenic (Mouse Insulin Promoter: MIP-hCXADR Tg) mice that express the human coxsackie and adenovirus receptor (hCXADR) gene specifically in pancreatic beta cells. In addition, the present embodiment has also confirmed that multiple lines of transgenic (Mouse Insulin Promoter: MIP-hCXADR Tg) mice that can be classified into three categories of low, moderate, and high expression levels can be produced.

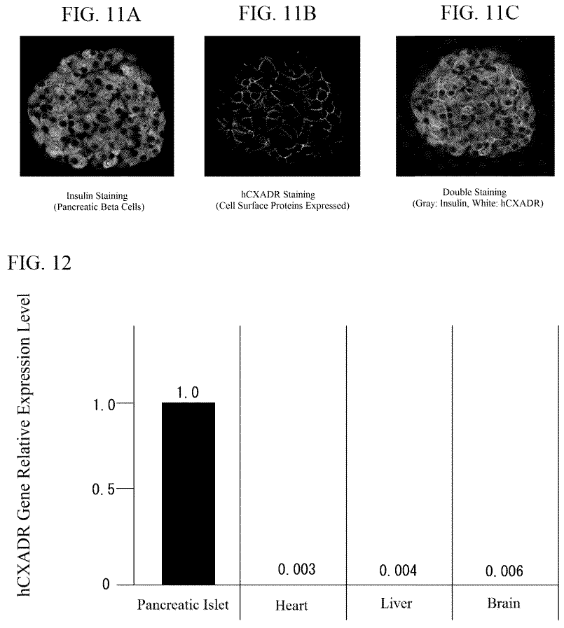

FIGS. 11A to 11C show results of immunostaining of hCXADR-positive mouse pancreatic islets infected with the coxsackie B4 virus. FIG. 11A shows a result of insulin staining (pancreatic beta cells), FIG. 11B shows a result of hCXADR staining (cell surface proteins expressed), and FIG. 11C shows a result of double staining (gray: insulin, white: hCXADR). From the obtained tissue findings, it was confirmed that the virus receptor protein (hCXADR) was on the surface of the pancreatic beta cells and the double staining caused the hCXADR protein to be expressed in the pancreatic beta cells.

### Example 2

### (Study of Pathology of Diabetes in Mice)

FIG. 12 shows a result of measuring relative expression levels of the human coxsackie and adenovirus receptor (hCXADR) gene introduced into the transgenic mouse according to Example 1 for each mouse organ. This is data of detection of a gene expression level in the mice with the hCXADR gene successfully introduced, by quantitative gene amplification method (quantitative PCR method), for each organ, and shows relative expression levels while assuming that the expression level for pancreatic islets is 1.0. The obtained results indicates that values themselves of the relative expression levels of the human coxsackie and adenovirus receptor (hCXADR) gene are too low for organs other than the pancreatic islet, and these values themselves have little meaning and are negligible as can be determined to be at <0.01. From this indication, it was confirmed that almost no expression in organs other than pancreatic islets occurred. Thus, it was confirmed that the human coxsackie and adenovirus receptor (hCXADR) gene was islet (organ)-specifically expressed in the pancreatic beta cells of the mouse pancreatic islet compared with other organs such as mouse heart, liver, and brain.

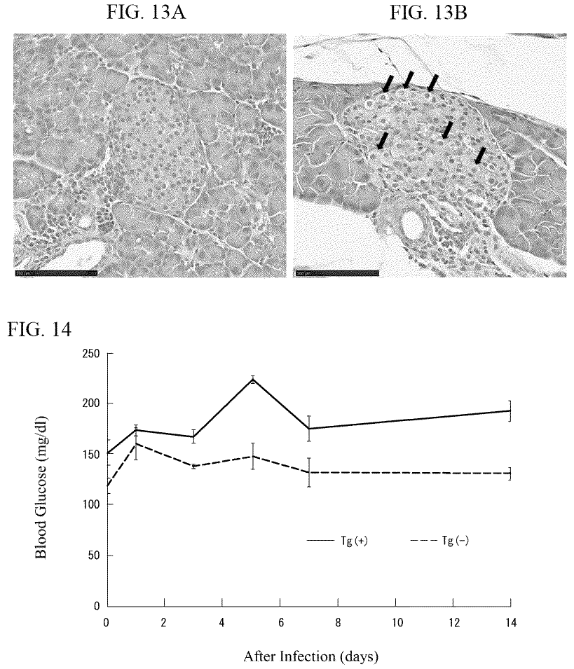

FIGS. 13A and 13B show resultant images of pancreatic islet tissue seven days after intraperitoneal inoculation of coxsackie B4 virus (10⁷ p.f.u.*) for the mouse (line 5) expressing the human coxsackie and adenovirus receptor (hCXADR) gene at a high level specifically to this pancreatic beta cells. Control wild-type mice that do not express the human coxsackie and adenovirus receptor (hCXADR) observed almost no islet cell damage, as shown in the result of FIG. 13A. On the other hand, the mice that express the human coxsackie and adenovirus receptor (hCXADR) gene at a high level specifically to pancreatic beta cells observed that swelling and degeneration of pancreatic islet cells occurred, as shown in the result of FIG. 13B (see arrows), which have revealed that the pancreatic islet cells were damaged (*p.f.u.: plaque forming unit; virus infectivity titer).

### Example 3

### (Study of Pathology of Diabetes in Mice)

FIG. 14 shows a measurement result of a blood glucose level (mg/dl) of the transgenic mouse obtained in Example 1 mentioned above for 14 days after such transgenic mouse was attacked (infected) with the coxsackie B4 virus. FIG. 14 shows an average value±standard error of blood glucose level for each legend of hCXADR (Tg+) n=3 and hCXADR (Tg−) n=4. From the obtained result, it was confirmed that the transgenic mouse had its blood glucose level significantly increased after infection with the coxsackie B4 virus. That is, Example 3 has revealed that the transgenic mouse obtained in Example 1 mentioned above establishes infection with the coxsackie B4 virus via the virus receptor expressed in pancreatic beta cells and this viral infection damages pancreatic beta cells to increase blood glucose.

### Example 4

### (Validation of Vaccine Effect)

The inventors experimentally confirmed protection of the coxsackie B virus vaccine against infected pancreatic islet damage at a cellular level. The inventors confirmed a preventive effect of the vaccine in mice attacked with a coxsackie B1 virus, one of the coxsackie B viruses, having an amount of virus of 10⁶ pfu.

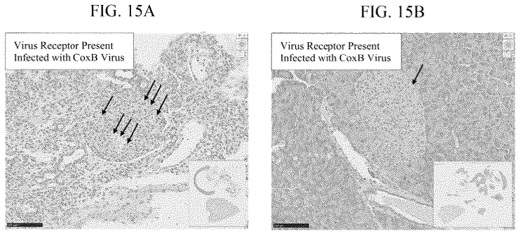

The results obtained are shown in FIGS. 15A and 15B. Infected pancreatic islets of a mouse infected with coxsackie B virus without administration of vaccine as a comparative example recognized many swellings and destructions (with inflammatory cell infiltration) (see arrows in the figure) in pancreatic islet cells, as shown in FIG. 15A. On the other hand, infected pancreatic islets of a mouse infected with the coxsackie B virus after formalin-inactivated coxsackie B virus vaccination recognized almost no swelling or destruction of pancreatic islet cells as appeared in the comparative example of FIG. 15A, as shown in FIG. 15B, confirming that inflammatory cell infiltration did not occur. From these results, an excellent ability of the coxsackie B virus vaccine to protect against virus-infected pancreatic islet damage was experimentally confirmed.

As described above, the viral diabetes high-susceptible mouse that successfully mimicked the mechanism leading to the onset of diabetes due to viral infection in humans was created. Using this mouse, it was confirmed that viruses causing diabetes can be identified with high sensitivity. This leads to the development of viral diabetes preventive vaccines in accordance with medical and scientific evidence, thus enabling prevention of viral diabetes. Such viral diabetes preventive vaccines can be any vaccines obtained by various known methods, and are not limited. Such vaccines include inactivated vaccines using, for example, formalin, phenol, ultraviolet light, and heat, gene vaccines such as messenger RNA (mRNA) vaccines and DNA vaccines, and virus like particle (VLP) vaccines which are considered highly safe. Promoting development of such viral diabetes preventive vaccines can lead to suppression of the incidence of both type 1 and type 2 diabetes patients, thus not only medical contribution but also social contribution being expected.

Regarding research ethics related to each of the above Examples, experiments to introduce the human coxsackie and adenovirus receptor (hCXADR) gene into mice (i.e., production of transgenic mice) obtained the approval from the Minister of Education, Culture, Sports, Science and Technology (30 received, Bunkashin No. 555), and the coxsackie and adenovirus receptor (hCXADR)-positive mice received the approval for its rearing and breeding at the animal experimentation facility of the Saga University.

### SEQUENCE LISTING

International patent application SU2207 PCPT transgenic mouse JP23005817
20230217----00160235352300348208 normal
20230217165214202212201138471370_P1AP101_SU_0.xml

1. A transgenic mouse, having DNA that encodes a human coxsackie group B virus receptor in pancreatic beta cells and expressing the human coxsackie group B virus receptor specifically in the pancreatic beta cells.

2. The transgenic mouse according to claim 1, wherein the DNA is placed under control of a mouse insulin promoter.

3. The transgenic mouse according to claim 1 or 2, wherein the human coxsackie group B virus receptor is a human coxsackie and adenovirus receptor (hCXADR) or a human decay accelerating factor (DAF).

4. The transgenic mouse according to claim 3, wherein the DNA is composed of:
a polynucleotide comprising a nucleotide sequence represented by SEQ ID NO: 1, or
a polynucleotide having a nucleotide sequence identity of 90% or more to the polynucleotide comprising the nucleotide sequence represented by SEQ ID NO: 1, and encoding a protein expressing the human coxsackie group B virus receptor specifically in the pancreatic beta cells.

5. The transgenic mouse according to any one of claims 1 to 4, obtained by injecting, into a fertilized egg, a plasmid vector composed of a mouse insulin promoter comprising a linear DNA fragment containing an SV40 polyA signal and a human coxsackie and adenovirus receptor (hCXADR).

6. The transgenic mouse according to any one of claims 1 to 5, wherein human coxsackie group B viral infection causes pathology of diabetes.

7. The transgenic mouse according to claim 6, wherein the diabetes is type 1 diabetes or type 2 diabetes.

8. A method for producing a diabetes preventive vaccine, using the transgenic mouse according to any one of claims 1 to 7.

9. The method for producing the diabetes preventive vaccine according to claim 8, the method comprising the steps of:
inoculating a candidate virus into the transgenic mouse;
determining an onset state of diabetes based on variation in blood glucose level and/or insulin level of the transgenic mouse inoculated with the candidate virus;
identifying an inoculation state of the candidate virus determined to have caused diabetes; and
producing the diabetes preventive vaccine based on the onset state and the inoculation state.

10. A method for screening a diabetes therapeutic drug, using the transgenic mouse according to any one of claims 1 to 7.


SEQUENCE LISTING (1 sequence)

SEQ ID NO: 1 - DNA, 1098 bp, Mus musculus
  source 1..1098: mol_type = genomic DNA
atggcgctcctgctgtgcttcgtgctcctgtgcggagtagtggatttcgccagaagtttgagtatcactactcctgaagagatgattgaaaaagccaaaggggaaactgcctatctgccatgcaaatttacgcttagtcccgaagaccagggaccgctggacatcgagtggctgatatcaccagctgataatcagaaggtggatcaagtgattattttatattctggagacaaaatttatgatgactactatccagatctgaaaggccgagtacattttacgagtaatgatctcaaatctggtgatgcatcaataaatgtaacgaatttacaactgtcagatattggcacatatcagtgcaaagtgaaaaaagctcctggtgttgcaaataagaagattcatctggtagttcttgttaagccttcaggtgcgagatgttacgttgatggatctgaagaaattggaagtgactttaagataaaatgtgaaccaaaagaaggttcacttccattacagtatgagtggcaaaaattgtctgactcacagaaaatgcccacttcatggttagcagaaatgacttcatctgttatatctgtaaaaaatgcctcttctgagtactctgggacatacagctgtacagtcagaaacagagtgggctctgatcagtgcctgttgcgtctaaacgttgtccctccttcaaataaagctggactaattgcaggagccattataggaactttgcttgctctagcgctcattggtcttatcatcttttgctgtcgtaaaaagcgcagagaagaaaaatatgaaaaggaagttcatcacgatatcagggaagatgtgccacctccaaagagccgtacgtccactgccagaagctacatcggcagtaatcattcatccctggggtccatgtctccttccaacatggaaggatattccaagactcagtataaccaagtaccaagtgaagactttgaacgcactcctcagagtccgactctcccacctgctaaggtagctgcccctaatctaagtcgaatgggtgcgattcctgtgatgattccagcacagagcaaggatgggtctatagtatag